(19) United States
(12) Patent Application Publication
(10) Pub. No.: US 2024/0200144 A1
(43) Pub. Date: Jun. 20, 2024

(54) METHODS AND APPARATUS UTILISING LIQUID BIOPSY TO IDENTIFY AND MONITOR PHARMACODYNAMIC MARKERS OF DISEASE

(71) Applicant: CERTARA USA, INC., Princeton, NJ (US)

(72) Inventors: Brahim ACHOUR, Manchester (GB); Amin ROSTAMI-HODJEGAN, Manchester (GB)

(21) Appl. No.: 18/431,820

(22) Filed: Feb. 2, 2024

Related U.S. Application Data
(63) Continuation of application No. PCT/EP2022/072490, filed on Aug. 10, 2022.

(30) Foreign Application Priority Data
Aug. 10, 2021 (GB) 2111504.3

(51) Int. Cl. C12Q 1/6886; A61K 45/06; C12Q 1/6806

(52) U.S. Cl. CPC C12Q 1/6886; A61K 45/06; C12Q 1/6806; C12Q 2600/106; C12Q 2600/158

(57) ABSTRACT

Methods and systems for generating a personalised pharmacodynamic profiles for an individual subject are provided. The method comprises the steps of: isolating total cell free RNA (cfRNATOTAL) from a liquid biopsy obtained from the individual subject and identifying an amount of at least a first cell free RNA (cfRNA) present in the liquid biopsy, wherein the first cfRNA encodes a first protein that has a pharmacodynamic activity. From the amount of cfRNA in the liquid biopsy an amount of the first protein in the individual subject can be determined, thereby allowing a personalised pharmacodynamic profile for the individual subject to be generated. The methods and systems find utility in precision dosing and personalised medicine.

This application is a continuation of International Application No. PCT/EP2022/072490, filed on Aug. 10, 2022, which claims priority to Great Britain Application No. 2111504.3, filed on Aug. 10, 2021, each of which is incorporated by reference herein in its entirety.

### FIELD OF THE INVENTION

The present invention is directed towards physiologically-based pharmacokinetic (PBPK) simulation systems, methods and apparatus for the modelling of pharmacodynamic interactions of drugs, toxins and other substances within animals, such as humans.

### BACKGROUND OF THE INVENTION

Cell free nucleic acids are present in the bloodstream and include RNA, so-called ‘circulating RNA’, despite the typically very short half-life of RNA outside cells (El-Hefnawy et al., Clin Chem, 2004). RNA molecules of this nature therefore are indicated to be associated with lipids, such as vesicles and lipoproteins, to enable their survival. Circulating cell free RNA—termed ‘cfRNA’—includes mRNA, which can be enriched in microvesicles or exosomes released by cells. The collection of cfRNA from bodily fluids, such as blood, for diagnostic or prognostic purposes is referred to as liquid biopsy. The amount of cfRNA released into circulation varies between individuals with ‘fast shedders’ releasing a higher amount of RNA for the same amount of transcription of a particular gene in the originating organ or tissue when compared to that released by ‘slow shedders’.

Liquid biopsies comprising of cfRNA, such as mRNA, have been used with a shedding correction factor applied in order to determine tissue abundance of proteins and enzymes involved in drug absorption, metabolism and clearance (see International Patent Application Publication No. WO-2019/191297-A), thus circumventing the need for solid tissue biopsies to determine such measurements. This technology allows for personalised assessment of pharmacokinetics in any given individual and provides a more accurate way to establish safer and more efficacious drug dosages for individuals.

Physiological information as well as compound-specific physicochemical data can also be used to describe the complex transport and transformation processes of a chemical compound throughout the body of an individual subject and to simulate its in vivo performance. This in silico model approach consists of so-called organ and tissue modules, termed ‘compartments’, connected by a circuit of flowing blood, subdivided as arterial and venous blood. The properties of each compartment are described by a series of differential equations with physiological parameters to represent the system in an integrated and biologically meaningful manner and combined to form what is called a physiologically-based pharmacokinetic (PBPK) model. PBPK modelling in combination with classical population pharmacokinetic (popPK) model-based simulations and liquid biopsy technology can be used to modify dosing and dosage regimen of drugs (see International Patent Application No. PCT/US2020/052261). The use of pharmacokinetics for a particular drug or xenobiotic in a specified individual has led to the generation of Virtual Twin PBPK models of clearance and metabolism (see US Patent Application No 2016/0335412).

Liquid biopsy approaches have centred around the issue of drug clearance and metabolism. Wider efforts to develop liquid biopsy approaches further, have been limited to specific contexts such as that described in Rowland et al. (Br J Clin Pharmacol. 2019 January; 85(1):216-226). In this study, the authors sought to demonstrate the presence of the clearance proteins CYP3A4 and UDP-glucuronosyltransferases (UGT) respectively and mRNAs in isolated human plasma exosomes and evaluate the capacity for exosome-derived biomarkers to characterize variability in CYP3A4 activity in patients treated with midazolam.

From a diagnostics standpoint, liquid biopsy approaches have been used to interrogate circulating cell free DNA liberated from tumours (Lanman et al. PLoS One, 2015, Oct. 16). This approach allows for genotyping of tumours via plasma-derived DNA instead of taking more invasive routes to obtain a solid tissue biopsy. Similar approaches are described in International Patent Application No: PCT/US2019/058380, published as WO-2020/092259-A1, in which cell free mRNA is isolated from blood samples and used to identify biomarkers that may optionally provide pharmacokinetic information of disease status or treatment, particularly for bone marrow cancers such as multiple myeloma and acute myeloid leukaemia. It is notable that in PCT/US2019/058380 no indication is made to correlate the cell free RNA levels for a given biomarker with abundance of the corresponding protein either systemically or locally. Hence, there remains a need to extract additional value from the liquid biopsy approach. Maximising the technical application of information derived from liquid biopsy would be of substantial benefit in further improving the design of clinical trials and the practical implementation of precision medicine.

These and other uses, features and advantages of the invention should be apparent to those skilled in the art from the teachings provided herein.

### SUMMARY OF THE INVENTION

Accordingly, the present inventors have identified that the liquid biopsy approach may be expanded beyond the conventional considerations of xenobiotic clearance, transport and metabolism, such that identification and characterisation of additional pharmacodynamic markers in plasma may provide improved applications in precision medicine. In particular, liquid biopsy assessment of cfRNA that corresponds to relevant pharmacodynamic markers in an individual may provide important diagnostic or prognostic intelligence in relation to homeostasis, particular cellular functions, disease progression or even acute or chronic pathologies.

A first aspect of the invention provides for a method of generating a personalised pharmacodynamic profile for an individual subject, the method comprising the steps of:
isolating total cell free RNA (cfRNA_{TOTAL}) from a liquid biopsy obtained from the individual subject;
identifying an amount of at least a first cell free RNA (cfRNA) present in the liquid biopsy, wherein the first cfRNA encodes a first protein that has a pharmacodynamic activity;
determining an amount of the first protein in the individual subject; and
generating a personalised pharmacodynamic profile for the individual subject.

In a specific embodiment of the invention, determining the amount of the first protein comprises the normalisation of the amount of the first cfRNA to account for cfRNA shedding in the individual subject. Suitably, the normalisation is made with reference to one or more tissue specific markers present in the cfRNA_{TOTAL}. Typically, the normalisation is made with reference to a plurality of tissue specific markers present in the cfRNA_{TOTAL}. Optionally, normalisation is made with reference to the amount of cfRNA_{TOTAL} present in the liquid biopsy sample.

In a specific embodiment the method of the second aspect comprises identifying amounts of a plurality of cfRNAs in order to generate a pharmacodynamic profile for the individual subject that comprises the amounts of the plurality of proteins that have a pharmacodynamic activity.

Optionally, all or a part of the methods described herein are implemented via one or more computer systems.

Accordingly, in a second aspect of the invention there is provided a computer implemented process for generating a personalised pharmacodynamic model for an individual subject who has a disease, the process comprising the steps of:
i. identifying at least a first pharmacodynamic marker for the disease, wherein the first pharmacodynamic marker is a gene that is expressed as a first mRNA;
ii. isolating total cell free RNA (cfRNA_{TOTAL}) from a liquid biopsy obtained from the individual subject;
iii. analysing the cfRNA_{TOTAL} to determine an amount of the first mRNA present in the liquid biopsy, thereby determining an amount of the first pharmacodynamic marker in the individual subject; and
iv. generating a personalised pharmacodynamic model for an individual subject based upon the amount of the first pharmacodynamic marker present in the individual subject.

A third aspect of the invention, provides a system for providing a treatment recommendation for an individual subject who has a disease, the system comprising:
an input device, for inputting data relating to the subject;
a computer readable medium containing program instructions for implementing a method of as described according to the third aspect, wherein execution of the program instructions results in one or more processors of the system carrying out the method; and
an output device for presenting the treatment recommendation based upon the amount of a first pharmacodynamic marker present in the individual subject.

A fourth aspect of the invention provides a method of treating an individual subject, wherein the individual is the intended recipient of a pharmaceutical treatment, the method comprising establishing a personalised pharmacodynamic profile for the individual subject prior to or during the treatment, the method comprising the steps of:
isolating total cell free RNA (cfRNA_{TOTAL}) from a liquid biopsy obtained from the individual subject;
identifying an amount of a first cell free RNA (cfRNA) present in the liquid biopsy, wherein the first cfRNA originates from a specified compartment within the body of the subject, and wherein the first cfRNA encodes a protein from the compartment that has a pharmacodynamic activity relevant to the pharmaceutical treatment;
determining a pharmacodynamic activity relevant to the pharmaceutical treatment for the individual subject based upon the presence or absence, or a level of the protein within the specified compartment of the subject;
generating the personalised pharmacodynamic profile for the individual subject; and
treating the individual according to a dosage regimen for the pharmaceutical treatment that is optimized to the individual based upon their personalised pharmacodynamic profile.

In a specific embodiment, the pharmaceutical treatment comprises administration of a xenobiotic; suitably the xenobiotic is a pharmaceutical agent, such as a pharmaceutical compound (e.g. a small molecule), biological or drug.

Typically, the liquid biopsy comprises a sample of a bodily fluid selected from one of the group consisting of: blood; urine; saliva; semen; tears; sweat; lymphatic fluid; bile; cerebrospinal fluid; ascites; pleural effusion; stool; and a mucus secretion. In particular embodiments the liquid biopsy comprises whole blood, serum and/or plasma.

Optionally, the methods of the first aspect comprise identifying at least a first and a second cfRNAs; suitably identifying a plurality of cfRNAs each corresponding to a different compartment protein.

In specific embodiments of the invention the individual subject has a disease selected from one or more of the group consisting of: cancer; liver disease; inflammatory disease; allergy; metabolic diseases, including metabolic deficiency; degenerative diseases, including neurodegenerative diseases; psychiatric disorders; infection, including chronic or acute infection from bacterial, viral, fungal or parasitic pathogens; autoimmune disease; kidney disease; anemia; heart disease; myocardial infarction; obesity; fibrosis; and traumatic brain or CNS injury.

It will be appreciated that the features of the invention may be subjected to further combinations not explicitly recited above.

### BRIEF DESCRIPTION OF DRAWINGS

The invention is further illustrated by reference to the accompanying drawings in which:

FIG. 1 shows an illustration of mRNA shedding from an organ in exosomes into circulating blood, in this instance the liver is shown as the tissue of origin. The targets are the RNA species of interest, including enzymes, transporters and pharmacodynamic markers. Tissue-specific markers are used to monitor shedding in each individual specific to the organ of origin into the bloodstream.

FIG. 2A shows a representation of identified pharmacodynamic markers as mRNA in plasma and as corresponding protein in liver tissue. The expression of identified markers in liquid biopsy spanned nearly five orders of magnitude.

FIG. 2B shows the measured plasma levels of three ubiquitously expressed pharmacodynamic markers in liver cancer patients correlated to the quantified protein abundance levels of these markers in matched organ tissue (liver). The levels in plasma were not normalized to tissue-specific shedding because these markers originate from multiple organs (compartments) including the liver. The expression of these markers is therefore only normalized to total shedded cfRNA in the plasma of the individual patients. The three pharmacodynamic markers are epidermal growth factor receptor (EGFR), interleukin enhancer-binding factor 3-A (ILF3), and dipeptide peptidase 4 (DPP4).

FIG. 2C shows the measured plasma expression levels of two tissue-specific pharmacodynamic markers in liver cancer patients correlated to the quantified protein abundance levels of these markers in matched organ tissue (liver). Plasma RNA levels have been adjusted to account for liver-specific shedding. The two pharmacodynamic markers are alanine aminotransferase 1 (GPT) and galectin 4 (LGALS4).

FIG. 3A shows differential expression of pharmacodynamic and disease markers in liquid biopsy from liver cancer patients relative to healthy control. Significant changes from healthy expression are used to identify useful markers of disease and treatment. In this instance sixteen pharmacodynamic markers were upregulated in liver cancer patients and seven were downregulated.

FIG. 3B shows the association between the differentially expressed pharmacodynamic markers and survival prognosis of liver cancer patients.

FIG. 4 shows a flow chart describing one embodiment of the invention.

### DETAILED DESCRIPTION OF THE INVENTION

Unless otherwise indicated, the practice of the present invention employs techniques of chemistry, computer science, statistics, molecular biology, microbiology, recombinant DNA technology, and chemical methods, which are within the comprehension of a person of ordinary skill in the art. Such techniques are also explained in the literature, for example, T. Cormen, C. Leiserson, R. Rivest, 2009, Introduction to Algorithms, 3rd Edition, The MIT Press, Cambridge, MA; L. Eriksson, E. Johansson, N. Kettaneh-Wold, J. Trygg, C. Wikstom, S. Wold, Multi- and Megavariate Data Analysis, Part 1, 2nd Edition, 2006, UMetrics, UMetrics AB, Sweden; M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

An embodiment of the present invention provides a modelling and simulation based system, which integrates transcriptomics (e.g. RNAomics) information from liquid biopsy with in silico, in vitro, and in vivo preclinical data from a wide range of sources with mechanism-based models to anticipate and predict the pharmacodynamic effects of xenobiotic molecules in humans or animals. The model utilises empirical and descriptive algorithms to describe the linkage between drug—or other xenobiotic—concentration, together with an observed response in various body tissues and/or compartments on drug response and potency, especially in organs such as the liver, kidney, brain/CNS, lungs or gut.

Prior to setting forth the invention, definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, subtypes of these such as genomic DNA, mRNA, miRNA, tRNA and rRNA. In embodiments of the invention mRNA is isolated from a liquid biopsy sample. Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers or probes for use in manipulation or detection of DNA such as via polymerase chain reaction (PCR).

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties that are characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “polypeptide” is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains.

As used herein the term “biomarker” or “response biomarker” may comprise cells, cellular components, peptides, polypeptides, proteins, ncRNA, genomic DNA, metabolites, cytokines, antigens, and polysaccharides; as well as physiological parameters such as blood pressure, pupil diameter, cell count, temperature, O₂ level, CO₂ level, pH, HbA1c level, serum potassium, serum creatinine, ejection fraction, heart rate, or QT interval. Suitably, the biomarkers comprise a combination of features. Such biomarkers can be important parameters in drug development and clinical trials. They may be used as readouts to measure the level of response to a therapeutic intervention and to guide clinical dose-response studies.

The term “levels” is used herein to define terms of quantity or abundance of a specified factor and may be defined in molar or absolute amounts (i.e. micrograms or milligrams etc.), concentration (e.g. mg ml⁻¹ or mol g⁻¹ etc.), and/or in terms of a specific activity (e.g. units of activity in a standard assay). The selected “level” will be appreciated as appropriate to a given factor, for example, where it is appropriate to define the amount of a given enzymatic factor by its specific activity, it may be that this measure is selected rather than the actual amount (in mg/ml) of that factor that may be present. In a specific embodiment, a level corresponds to the amount of a specified protein per unit of tissue—for example, in moles per unit mass of tissue. The term “normal level”, when in the context of levels of gene or polypeptide expression, is used herein to denote the level of gene expression or enzymic activity in healthy non-diseased organs, tissue, compartments or samples. Normal levels of expression or activity represent the baseline or control level of expression of a gene. Aberrant levels in cells, either at levels that exceed the normal range or that are too low, are considered not to be normal and can be indicative of disease in the samples from which the cells have been obtained, e.g. cancer. The term “high” in relation to levels may refer to strong, consistent and/or readily detectable expression and may not be considered as necessarily aberrant.

The term “allelic variant” is used herein to denote any two or more alternative forms of a gene occupying the same chromosomal locus and controlling the same inherited characteristic. Allelic variation arises naturally though mutation, and may result in phenotypic polymorphism within populations. Gene mutations typically result in an altered nucleic acid sequence and in some cases an altered polypeptide sequence also. As used herein, the term “allelic variant” is additionally used to refer to the protein or polypeptide encoded by the allelic variant of a gene.

The term “isolated”, when applied to a polynucleotide sequence, denotes that the sequence has been removed from its natural organism of origin and is, thus, free of extraneous or unwanted coding or regulatory sequences. The isolated sequence is suitable for use in recombinant DNA processes and within genetically engineered protein synthesis systems. Such isolated sequences include cDNAs and genomic clones. The isolated sequences may be limited to a protein encoding sequence only (e.g. an mRNA), or can also include 5′ and 3′ regulatory sequences such as promoters, transcriptional terminators and UTRs.

The term “isolated”, when applied to a polypeptide is a polypeptide that has been removed from its natural organism of origin. Typically, the isolated polypeptide is substantially free of other polypeptides native to the proteome of the originating organism. Suitably, the isolated polypeptide may be in a form that is at least 95% pure, more suitably greater than 99% pure. In the present context, the term “isolated” is intended to include the same polypeptide in alternative physical forms whether it is in the native form, denatured form, dimeric/multimeric, glycosylated, crystallised, or in derivatized forms.

As used herein, the term “organ” is synonymous with an “organ system” and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver, kidney, brain, gut or pancreas; or may comprise fluid organ systems such as the blood and circulatory system. Typically organs comprise at least two tissue types, and/or a plurality of cell types that exhibit a phenotype characteristic of the organ. In contrast “tissue” refers to an aggregation or population of cells of the same or a similar type and/or lineage that may cooperate with other tissues to form an organ system.

The term “compartment” as used herein refers to the concept of a tissue or organ system compartment as often used in pharmacokinetic modelling (for example, see Thompson & Beard J Pharm Sci. 2012 January; 101(1): 424-435). Compartments are typically defined as comprising organs and/or tissues that are interlinked anatomically and/or physiologically and comprise specified volumes, perfusion rates and tissue connectivity. The nature of a compartment allows for rate of absorption, distribution, metabolism, and excretion (ADME) of a drug or xenobiotic compound to be determined at a mechanistic level based upon the biochemical and biophysical characteristics of the tissues comprised within the compartment. In some instances, an organ may be regarded as comprising multiple compartments that reflect the complexity of its functions. By way of example, the liver may be represented as having a single compartment or as a plurality of individual compartments, some of which have a variable perfusion rate which can be used to account for the complex architecture of the hepatic vasculature. In other instances, a compartment may be defined as encompassing more than one organ system. In the context of the present invention, a compartment may be used to define one or more tissues that comprise the site of action of a given drug or xenobiotic, and which tissues cooperate at a pharmacodynamic and/or pharmacokinetic level to influence the concentration of the given drug or xenobiotic at the said the site of action.

The term “sample” is used to describe isolated materials of biological origin that can be used for a diagnostic, analytical or prognostic purpose. Biological materials may be analysed in tissue microarrays, or via other assay methods, and can include tissues from specific organs such as liver, kidney, brain, heart, epithelium, lung, and bone, as well as other tissues; as well as fluid materials such as whole blood, plasma, serum, sweat, lymph, urine, stool, cerebrospinal fluid, ascites, pleural effusion and saliva etc. Such materials may also include in vivo and in vitro cellular materials such as healthy or diseased cells, tissues and cell lines—e.g. cancer cell lines, which may be manipulated for in vitro purposes—e.g. immortalised cell lines or induced pluripotent stem cells. The macromolecules analysed in these materials typically include polypeptides such as proteins as well as polynucleotides such as RNA (including mRNA), and DNA.

The term “blood sample” may refer to any or all of whole blood, plasma, serum, erythrocyte and/or leucocyte fractions, and any other blood derivative. Blood samples may be comprised within a liquid biopsy obtained from an individual or plurality of individuals.

The term “microsome” refers to vesicles made by re-forming of the endoplasmic reticulum (ER) during the break-up of cells in vitro, which can be concentrated and isolated from other cell debris. Many pharmacodynamic markers are present in ER or are associated with other cellular membranes—e.g. as receptors—and so microsomal preparations containing such markers can be obtained from tissue samples such as organ tissue (e.g. liver) of specific compartments, where these pharmacodynamic markers are abundant. Examples of suitable pharmacodynamic markers are further discussed below.

The term “microvesicle” or “exosome” relates to extracellular vesicles that may be produced or shed by cells for example by exocytosis, budding or blebbing of the plasma membrane. Cell death by apoptosis may also lead to microvesicle production. Microvesicles are found in interstitial space and in many body fluids, and may contain mRNA, miRNA and/or proteins. It is thought that methods of intercellular communication may rely on microvesicle transport. Exosomes are a type of microvesicle that range in size from nanometer scale through to micrometer size. Exosomes are derived from parental cells comprised within organs or tissues so they are able to reflect both the physiological and pathophysiological state of those parental cells.

“Cell free nucleic acid” may be DNA, RNA, or any combination thereof. The nucleic acid may be cell free DNA (cfDNA), cell free RNA (cfRNA), or any combination thereof. The samples from which the cell free nucleic acids may be isolated include any bodily fluid capable of providing a liquid biopsy. Where the liquid biopsy comprises blood, the cell free nucleic acids may be located within plasma or serum.

As used herein the term “shedding” is used to describe the process of mRNA release by cells from organs or tissues, such as liver hepatocytes, into a bodily fluid, in microvesicles, exosomes, or otherwise as cell free mRNA. mRNA shedding can vary in magnitude between subjects or within the same subject depending on, for example, disease state, and affects the correlation between the levels of a particular RNA detected in the blood, plasma or other sample, and the levels of the same mRNA in the cells and tissue of the organ, such as the liver. The term “RNA shedding” is used as a synonym. As mentioned above, variable shedding between individuals of cfRNA can be corrected for in liquid biopsies, see International Patent Application Publication No. WO-2019/191297-A, which is incorporated herein by reference. When a particular pharmacodynamic marker is ubiquitously expressed, rather than derived from a specific organ or tissue, the normalization correction may be carried out relative to the total cell free RNA (cfRNA_{TOTAL}) shed by the individual present in the plasma.

Pharmacokinetics (PK) is the study of what happens to a drug when it is administered to and passes through the various organ and tissue compartments within the body of a subject. Drug absorption, distribution, and elimination are subject to multiple interactions dependent in part upon the biological action of each organ on a drug, partitioning of the drug to these organs and tissue volumes (compartments) and blood flows. The absorption (rate and extent of bioavailability), distribution/localisation, metabolism and excretion (ADME), biotransformation and toxicity profiles of any given pharmaceutical or other xenobiotic compound are key deterministic measures of subsequent pharmacodynamics (action of the drug on body) necessary to achieve efficacy without major safety issues prior to an authorisation for use in medicine.

Pharmacodynamics (PD) relates to the effects of the interaction of a drug or other xenobiotic compound with the tissues, organs, or compartments of the body. In particular aspects, pharmacodynamics is concerned with quantifying the relationship between the concentration of a given drug at the site of action and any resultant biochemical and physiological effects that are elicited. This is distinct from pharmacokinetics which is more concerned with how the tissues and compartments within the body transport, metabolise and excrete a given drug or compound. Both pharmacodynamics and pharmacokinetics show variability between individuals in a population. Variability between individuals in pharmacodynamic response may be due to genetics, environment, age, ethnicity or sex, or other factors such as the development of tolerance to the drug as a result of continued exposure. High levels of inter-individual variability in pharmacodynamic response are problematic for drug development as well as monitoring. As used herein, the phrases “pharmacodynamic marker” or “PD marker” includes genes that are determinative of a pharmacological response that is directly linked to engagement of the primary molecular target by a drug or xenobiotic compound. As such, the presence or absence of activity of pharmacodynamic markers may intervene upstream in the cascade of events that ultimately lead to a disease pathology. For this reason, pharmacodynamic markers are also sometimes referred to as proximal biomarkers. Pharmacodynamic markers, or the pathways in which they participate, are typically modulated by drug treatment in a drug concentration-dependent manner and hence, are able to correlate drug concentration at the site of action to target occupancy when not at saturation. In certain circumstances the pharmacodynamic marker may also represent the therapeutic target of a specified drug, for example if it is a cell surface receptor or DNA binding protein. In such an instance, the pharmacodynamic marker is referred to as a drug target. Determination of the levels of one or more pharmacodynamic markers for a specified disease, such as cancer, can also be useful in identifying factors that will influence clinical decisions. By way of example, pharmacodynamic markers of tumour growth, metastases or that indicate adverse effects to an administered treatment can provide useful prognostic information and may contribute to creation of personalised medicine approaches to cancer therapy.

For any given disease or condition there can be multiple pharmacodynamic markers that may be useful from a prognostic or diagnostic perspective. It will be appreciated that the present invention is directed towards methods and systems that are able to provide additional information about any specified pharmacodynamic marker that is comprised within cfRNA comprised within a liquid biopsy sample by enabling the determination of corresponding abundance of the RNA and/or the protein encoded by that RNA. This information can be used to inform in silico models generated for the individual patient and, therefore, simulations to be performed to improve personalised precision dosing strategies. Hence, the methods and systems described herein are not limited to particular diseases or to specified panels of pharmacodynamic marker genes but can readily be applied more broadly to any disease with any particular selection of pharmacodynamic markers as long as they are expressed and present as cfRNA within a liquid biopsy sample obtained from an individual subject.

In an embodiment of the present invention, cfRNA coding for one or more pharmacodynamic markers may be identified within a liquid biopsy obtained from an individual subject. In a specific embodiment of the invention, a method provides for the identification of cfRNA that codes for one or more than one, or a plurality, of pharmacodynamic markers and their site of origin determined. The site of origin may refer or correspond to a tissue, organ, organ system or compartment within the body of the individual subject. The level of the cfRNA of one or more pharmacodynamic markers may be quantified as a concentration, for example, as a number of transcripts per unit volume or per unit mass of the liquid biopsy sample, or extrapolated per the unit mass of the subject from which the sample was derived.

In one embodiment of the invention, a liquid biopsy sample of plasma is isolated from an individual subject. The liquid biopsy sample is processed to isolate cfRNA comprised within one or more exosomes comprised within the plasma. The cfRNA is separated and subjected to nucleic acid extraction prior to transcriptomic analysis (such as via high throughput next generation RNA sequencing). The transcripts present in the plasma sample can be quantified and identified by cross referencing with transcriptomic and gene expression databases using RNA-Seq bioinformatics approaches. Transcriptomic analysis may include identification of known pharmacodynamic targets and markers or may be used to identify novel—i.e. previously unknown—pharmacodynamic markers and targets. The disease status of the individual subject as well as other biomarker factors may be taken into account when assessing the pharmacodynamic information made available through the transcriptomic analysis of the liquid biopsy. By making a determination of a combination of parameters including relevant biomarkers, pathophysiology, disease status, administered drugs and or xenobiotic compounds, it is possible to assess the pharmacodynamic factors that may be important in assessment of drug choice, therapeutic strategies, therapeutic outcome, dosage regimen design and dosage levels choice. Such choices may inform how an individual subject is treated thereby leading to new methods of treatment for a given condition.

The approach described according to the present invention shows considerable advantage in areas which are currently intractable to drug discovery. In addition, the methods of the invention show particular advantage in conditions where the number of individual subjects suffering from a particular condition or disease is relatively small. This facilitates the trialing of orphan drugs or therapeutic approaches which otherwise would not be possible due to the small number of individuals available for clinical testing.

For specific clinical trials, rapid deployment or repurposing of existing drugs, an in depth understanding of pharmacodynamics is an essential factor that must be considered before deciding how to design a clinical trial. The present invention provides highly detailed structural and functional information around the pharmacodynamics at the site of action of a given drug or xenobiotic. Hence, studies that could have taken months or even years previously to determine the detailed pharmacodynamics of a new drug can be avoided by following the methodology set out herein.

According to a specific embodiment of the invention, one or more novel pharmacodynamic markers may be identified and associated with pathophysiology or disease status in an individual subject based upon a liquid biopsy sample analysis for cfRNA content. Data relating to one or more novel pharmacodynamic markers may be aggregated or otherwise collected in order to generate a consolidated population-based analysis. A population may be created from a plurality of individual subjects defined by, for instance, demographics, genetics, ethnicity, age or any other collective factor. The population may be used to create in silico modelling simulations that may be utilised in drug discovery or monitoring. Such in silico models may be supplemented with pharmacokinetic data and modelling capability—e.g. popPK or PBPK modelling.

In addition to the pharmacodynamic information, pharmacokinetic information may be derived from the same or a different liquid biopsy taken from the individual subject. Suitably, the transcriptomic analysis of the liquid biopsy sample may be extended to include pharmacokinetic markers as well as pharmacodynamic markers. Suitably, pharmacokinetic markers may include cytochrome P450 monooxygenase enzymes (CYPs) as well as membrane transport proteins, and transferases. In embodiments of the invention the CYP enzymes are selected from human CYP families 1, 2 and 3, which are the CYP families typically linked to xenobiotic (e.g. drug) metabolism and clearance. CYPs may be implicated in disease and can serve as pharmacodynamic markers. Suitably the CYPs may comprise any, some or all of the CYPs selected from the group consisting of: CYP1A1; CYP1A2; CYP1B1; CYP2A6; CYP2A7, CYP2A13; CYP2B6; CYP2C8; CYP2C9; CYP2C18; CYP2C19; CYP2D6; CYP2E1; CYP3A4; CYP3A5 and CYP3A7; it will be appreciated that this list is non-exhaustive. The CYP3A subclass catalyzes an extensive number of oxidation reactions of clinically important drugs. It is currently believed that greater than 60% of clinically used drugs are metabolized by the CYP3A4 enzyme, including several major drug classes. Hence, accurately determining the abundance of even CYP3A4 alone in the liver of an individual subject based upon a liquid biopsy would facilitate the development of PBPK models able to predict that individual's capacity for clearance of many approved drugs currently on the market. Such information can be used to supplement the pharmacodynamic analysis of the individual subject which focusses upon the presence of particular pharmacodynamic markers at the site of action of the drug. Hence, by understanding the combined pharmacodynamic and pharmacokinetic profile of the individual subject improved treatment approaches may be devised.

Other, non-CYP, proteins that are involved in metabolism of xenobiotic molecules and may act as pharmacodynamic or pharmacokinetic markers include transferases: enzymes that catalyse the transfer of a functional group from a donor molecule to a specified substrate molecule (an acceptor) which is typically a drug or other xenobiotic compound. Transferase enzymes involved in drug metabolism are typically those that catalyse conjugation of moieties such as glutathione, methyl groups, acetyl groups, sulfate, and amino acids to a substrate molecule which may be a drug or a metabolite of a drug. Exemplary drug metabolizing transferases may include methyltransferases; sulfotransferases; N-acetyltransferases; glucuronosyltransferases (UDP-glucuronosyltransferases or UGTs) including, but not limited to, one or more of the group consisting of UGT1A1, UGT1A3, UGT1A4, UGT1A6, UGT1A9, UGT2B4, UGT2B7, UGT2B15 and UGT2B17; glutathione-S-transferases; and choline acetyl transferases.

In addition to the above, membrane bound and non-membrane bound transport proteins also may influence the levels of xenobiotic (or metabolite) compound uptake and, hence, the levels of metabolism and clearance of a given compound within the body of an individual. Transport proteins may include one or more of the group selected from: transmembrane pumps, transporter proteins, escort proteins, acid transport proteins, cation transport proteins, vesicular transport proteins and anion transport proteins. Exemplary transporter proteins include ATP-binding cassette (ABC) transporters including, but not limited to, one or more of the group selected from: ABCB1/MDR1, ABCB11/BSEP, ABCC2/MRP2, ABCG2/BCRP. Alternatively, solute carrier (SLC) transporters may include one or more of the group consisting of: SLCO1B1/OATP1B1, SLCO1B3/OATP1B3, SLCO1A2/OATP1A2, SLCO2B1/OATP2B1, SLC22A1/OCT1, SLC22A7/OAT2, and SLC47A1/MATE1.

It will be appreciated that in certain instances a specified xenobiotic compound, molecule or composition may act as a substrate for several drug metabolizing enzymes and/or drug clearance proteins. It is an advantage of the present invention that a virtual PBPK model may be constructed that incorporates the relative contributions of a plurality of enzymes and/or proteins, such as those described herein, that are involved in the clearance and/or metabolism of the specified xenobiotic. The plurality of xenobiotic/drug clearance or metabolizing enzymes or proteins that inform the virtual PBPK model may comprise a plurality of CYPs, or a combination of one or more CYPs and one or more non-CYPs, suitably one or more CYPs and one or more transferases and/or transporters.

In an embodiment, the present invention is based in part upon an assay system or method that determines the presence and quantity of one or more mRNAs coding for pharmacodynamic markers originating systemically or from an organ/tissue in a liquid biopsy, such as a biological blood sample, via a quantitative analysis of the sample. This analysis establishes the pathophysiological status within a tissue, a compartment or the body as a whole of the individual who has been tested. By assessing the expression of one or more mRNAs coding for pharmacodynamic markers within the individual it is possible to create a personalised profile of that individual's health status, including diagnostic or prognostic information. This profile may be used to direct a personalised medicine approach.

In a specific embodiment of the invention, the assay system or method may further combine the presence of one or more pharmacodynamic markers with a personalised PBPK model for that individual. The personalised PBPK model may be selected according to the disease or health status of the individual subject. The PBPK model may be selected or constructed on the basis of cfRNA levels identified in the liquid biopsy and correlated to relative abundance or concentration of xenobiotic metabolizing and/or transporting proteins in an organ/tissue/compartment, or enzymes and/or transporters in other tissues of an individual, that are relevant to the pharmacodynamic context of the individual. The abundance relationship may be supplemented by reference to standardized curves or log tables generated by comparison of matched samples comprising a liquid biopsy and a tissue biopsy from one or more reference individuals. Generally, the matched samples are obtained from the same individual. In this way, the concentration of mRNA for a clearance or metabolizing protein present in the liquid biopsy is capable of direct relation to the corresponding abundance/concentration of the protein in the organ/tissue/compartment of origin. This in turn allows for the xenobiotic clearance capacity of the organ/tissue/compartment of origin to be estimated with a high degree of accuracy in the patient cohort, without the need for further sampling of solid tissue biopsies. Information regarding the xenobiotic clearance capacity of an organ or tissue for a given protein having pharmacokinetic activity (such as a CYP, transferase or transporter) represents one of the building blocks of bottom-up PBPK models. Hence, in this way the combination of a pharmacodynamic profile with relevant PBPK modelling analysis from a single liquid biopsy can inform drug dosage choices for the subject individual.

The individuals tested or treated according to various embodiments of the invention may be healthy or diseased, and human or animal patients. In veterinary contexts, the drug clearance models may require suitable adaptation, although the underlying principles of the invention are consistent. The term “animal” may include mammals such as cats; dogs; mice; guinea pigs; rabbits; primates; horses; as well as livestock including cattle; pigs; sheep; and goats.

For individuals or populations suffering from disease or abnormal pathology the current development of pharmacodynamic profiling is difficult due to a lack of available data to better characterize the altered physiological state. The lack of in vivo data for generating models for individuals and populations suffering from cancer is particularly evident. Prediction of drug response during infection and inflammation is a further important consideration for disease state modelling, given that this altered state leads to downregulation of metabolizing enzymes such as CYPs in the liver and gut due to elevated levels of pro-inflammatory cytokines. Since the design of personalised dosage regimens for drugs in individual patients has been hitherto assigned based on the extent of drug clearance for that individual to avoid overexposure, accounting for the effect of disease such as renal or hepatic impairment, which can significantly reduce clearance, is crucial. Hence, in embodiments of the invention a method is provided for establishing a virtual model of pharmacodynamic and pharmacokinetic response in an individual subject or a population of individual subjects, wherein the subject(s) are suffering from disease or altered physiological state associated with an abnormal pathology.

According to embodiments of the present invention personalised virtual models may be utilised in the development of more personalised dosage regimens. Such personalised dosage regimens may be used in methods of treatment of individual subjects in need thereof. In specific embodiments, dosage regimens may be formulated for the treatment of a range of diseases including, but not limited to, cancer; liver disease; inflammatory disease; allergy; metabolic diseases, including metabolic deficiency; degenerative diseases, including neurodegenerative diseases; psychiatric disorders; infection, including chronic or acute infection from bacterial, viral, fungal or parasitic pathogens; autoimmune disease; kidney disease; anemia; heart disease; myocardial infarction; obesity; fibrosis; and traumatic brain or CNS injury.

According to embodiments of the invention, cancer may include: carcinomas, leukemias, adenocarcinomas, gliomas, glioblastoma, brain metastases, multiple myelomas, renal clear cell carcinoma, prostate cancer, pancreatic adenocarcinoma, melanoma, metastatic melanoma, rhabdomyosarcoma, hepatocellular carcinoma, metastatic liver cancer, colon tumours, breast cancer, non-small cell lung cancer, oral tumours, colorectal cancer, gallbladder cancer, brain tumours, Ewing's sarcoma, bladder cancer, meningioma's, lymphoma, viral-induced tumours, Burkitt's lymphoma, Hodgkin's lymphoma, adult T-cell leukemia, lymphoproliferative disease, Kaposi's sarcoma, as well as MALT lymphoma, papillary thyroid carcinoma, cervical cancer, osteosarcoma; primary intra-ocular B-cell lymphoma, mesotheliomas, ovarian cancer, cervical cancer, head and neck cancer, small cell lung cancer, cancer of the oesophagus, stomach cancer, hepatobiliary cancer, cancer of the small intestine, rectal cancer, kidney cancer, bladder cancer, penile cancer, urethral cancer, testicular cancer, cervical cancer, vaginal cancer, uterine cancer, thyroid cancer, parathyroid cancer, adrenal cancer, pancreatic endocrine cancer, carcinoid cancer, bone cancer, skin cancer, retinoblastomas, non-Hodgkin's lymphoma, multicentric Castleman's disease or AIDS-associated cancer, primary effusion lymphoma, and neuroectodermal tumours.

According to embodiments of the present invention, inflammatory diseases may include: asthma, keratitis, rhinitis, stomatitis, mumps, pharyngitis, tonsillitis, tracheitis, bronchitis, pneumonia, myocarditis, gastritis, gastroenteritis, cholecystitis, and appendicitis.

According to embodiments of the invention autoimmune disorders may include chronic lymphocytic thyroiditis, hyperthyroidism, insulin-dependent diabetes mellitus, myasthenia gravis, chronic ulcerative colitis, pernicious anaemia associated with chronic atrophic gastritis, Goodpasture's syndrome, pemphigus vulgaris, pemphigoid, primary biliary cirrhosis, multiple cerebrospinal sclerosis, acute idiopathic neuritis, systemic lupus erythematosus, rheumatoid arthritis, psoriasis, systemic vasculitis, scleroderma, pemphigus, mixed connective tissue disease, autoimmune haemolytic anaemia, autoimmune thyroid disease, Crohn's disease and ulcerative colitis. Autoimmune disorders may further include transplant rejection such as comprising rejection of transplanted organs including kidney, liver, heart, lung, pancreas, cornea, and skin; graft-versus-host diseases brought about by stem cell transplantation; chronic allograft rejection and chronic allograft vasculopathy.

According to embodiments of the invention psychiatric disorders may include: dementia and Mild Cognitive Impairment (MCI); addiction; reduced adherence, or non-compliance, with a medication regime; eye gaze-associated disorders, dysthymia; psychotic disorders such as schizophrenia; eating disorders such as Anorexia Nervosa and Bulimia Nervosa; sleep disorders; developmental dyspraxia; attention deficit hyperactivity disorder; Tourette's syndrome, and personality disorders.

According to embodiments of the invention neurodegenerative diseases may include: Alzheimer (or Alzheimer's) disease, Parkinson's disease (including Parkinson's disease dementia), multiple sclerosis; adrenoleukodystrophy, AIDS dementia complex, Alexander disease, Alper's disease, amyotrophic lateral sclerosis (ALS), ataxia telangiectasia, Batten disease, bovine spongiform encephalopathy (BSE), Canavan disease, cerebral amyloid angiopathy, cerebellar ataxia, Cockayne syndrome, corticobasal degeneration, Creutzfeldt-Jakob disease (CJD), diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and neurodegenerative movement disorders.

According to embodiments of the invention fibrosis may include: liver cirrhosis, as well as idiopathic pulmonary fibrosis, renal fibrosis, endomyocardial fibrosis, and arthrofibrosis.

In an embodiment of the invention, the levels of mRNAs—suitably cell free mRNAs—that encode one or more pharmacodynamic markers, are measured in a liquid biopsy, suitably a blood sample. The concentration or amount of each mRNA in the blood sample thereby correlates to an amount/concentration/abundance of a drug clearance protein, for example, an enzyme or transporter, in the organ or tissue of the individual from which the mRNA originated. The prediction of amount/concentration/abundance of a drug clearance protein based upon the amount or concentration of the mRNA present in the liquid biopsy can be made by consultation with a calibration curve or log table, for instance.

The transcriptomics profile can be used to build a virtual system to provide an in silico model for an individual subject or if combined with a plurality of other individuals to provide a virtual population, or sub-population. Such models can be tested to predict the individual's or a population's capacity for clearance with one or more xenobiotic compounds. The system can be further refined by the addition of information derived from biomarkers found within the same or a different sample, and/or with other physiological and/or epidemiological information, which may be gathered by questionnaire, interview, health professional analysis, measurement with medical diagnostic equipment, or similar.

Isolation of exosomal or microvesicular components from a liquid biopsy may be performed using techniques such as spin column chromatography, immunoaffinity, membrane affinity, affinity labelled microbeads, precipitation and/or ultracentrifugation. Optimisation or choice of techniques will depend upon factors such as sample volume versus the type of liquid biopsy being handled. In an example of the invention described in more detail below, RNA comprised within exosomal or microvesicular components of a blood plasma liquid biopsy are isolated using a membrane affinity column utilising selective binding to a silica-based membrane.

Biomarker levels within a liquid biopsy sample may be determined by a range of techniques including macromolecule microarray analysis, mass spectrometry (MS) proteomic profiling, quantitative RT-PCR, ELISA or other antibody-based assays, and chromatographic or spectrophotometric techniques.

RNA transcripts that are isolated from the liquid biopsy sample may be detected by a range of methods, including but not limited to polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence detection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. RNA may be reverse-transcribed by any suitable means to produce cDNA before analysis in any combination with the above.

Bioanalysis of RNA samples may also occur using RNA sequencing such as by use of a single-end sequencing-by-synthesis reaction e.g. Ampliseq (Thermo Fisher, USA), HiSeq 2500 or NextSeq 550Dx Systems (Illumina, USA).

DNA arrays are solid supports upon which a collection of gene-specific nucleic acids have been placed at defined locations. In array analysis, a nucleic acid-containing sample is labelled and then allowed to hybridise with the gene-specific targets on the array. Based on the amount of nucleic acid from the sample hybridised to target on the array, information is gained about the specific nucleic acid composition of the sample. Array analysis, according to the present invention, involves isolating total RNA from a sample comprising cells or microvesicular material, converting the RNA samples to labelled cDNA via a reverse transcription step, hybridising the labelled cDNA to identical arrays (such as via either a nylon membrane or glass slide solid support), removing any unhybridised cDNA, detecting and quantitating the hybridised cDNA, and determining the quantitative data (e.g. the levels of biomarkers present) from the various samples.

Real-time or quantitative PCR refers to a method which monitors the replication of a nucleotide sample in real-time during the PCR reaction. As well as the normal components, the reaction mixture contains fluorescent probes which may hybridise to any double-stranded nucleotide sequence or else to a specifically chosen complementary sequence. The signal from the fluorescent probes therefore correlates with the number of the target sequences which have been produced during the reaction and can be used to determine the quantity of the target sequence in the original sample.

Additional factors may have a bearing on drug response as determined by analysis of the pharmacodynamics. These characteristics may be determined by the measurement of biomarkers in a sample, which can be the same or different sample as the liquid biopsy sample used for determination of the one or more cfRNAs. For example, allelic variations of pharmacodynamic markers, or any other relevant gene, may be determined from genomic DNA isolated from a liquid biopsy sample or any of a number of biological samples. This can include information not able to be derived from mRNA sequences, such as intron data, epigenetic information and the presence and activity of genomic regulatory features such as promoters, repressors, and so on.

Non-gene expression parameters which may also be relevant for determining drug response or dosage may include parameters which can be determined by measurement of biomarkers in one or more liquid biopsy sample, and/or can include physiological and epidemiological information collected by other means. In some embodiments of any aspects of the invention, one or more non-gene expression parameters may be selected from the group consisting of: ethnicity; genotype; age; age group classification; gender; smoking status; presence of chronic disease, including renal impairment, diabetes (type 1 or type 2) or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; general medical history; familial medical history; or combinations thereof. Such additional parameters may be used to further refine any model, algorithm, simulation or prediction produced by the invention, improving accuracy.

Embodiments of the present invention provide a method that is used to build a robust computer (in silico) predictive model of pharmacodynamic response, for a specified individual subject. In this way a computer-based model of drug response can be matched to any given individual, following a simple blood test, and thereby provides an accurate personal prediction of an individual's suitability for a given drug, xenobiotic, or combination of drugs or xenobiotics. This may be incorporated into a so-called Virtual Twin model, that also comprises pharmacokinetic information, which in turn is incorporated into a computer implemented system that can be utilised by, for example, clinicians, academics, patients and pharmaceutical researchers.

According to an embodiment of the invention the method comprises the steps of obtaining a liquid biopsy sample from an individual. The liquid biopsy may suitably comprise a bodily fluid such as any one or more of: blood, urine, saliva, semen, tears, sweat, lymphatic fluid, cerebrospinal fluid, bile, stool or a mucus secretion. This sample can be obtained via a minimally invasive route, and can include deriving blood components such as plasma, serum or other sample from a whole blood liquid biopsy sample. The sample is analysed to identify one or more, typically a plurality, of mRNAs coding for pharmacodynamic proteins in order to derive a profile for the said individual.

In some embodiments the method further comprises quantitatively analysing a sample to determine the levels of one or more, typically a plurality, of biomarkers present within the sample in order to derive a profile of the said individual's biomarker(s). The sample may be the same or different to that sample for determining circulating RNA, and as such may further include the steps of obtaining a second biological sample from an individual. The sample may be obtained in any suitable way, but may again be obtained via a minimally invasive route, such as a blood, cheek swab, saliva, stool or urine sample. The profile defines biomarker input data, which biomarker input data is used to calibrate a computer-based model of drug response.

In some embodiments, physiological and/or epidemiological information to obtain non-gene expression data not derivable from sample biomarkers may be obtained from an individual, in order to derive a physiological and/or epidemiological profile of the said individual. Such information may include ethnicity; age; gender; smoking status; body mass index (BMI); body adiposity index (BAI) or other equivalent measurements of body fat content; waist circumference measurement; waist-to-hip ratio; allergy status; blood pressure; average resting heartbeat; ECG interval measurements including QT interval, QRS duration, and PR intervals; general medical history; familial medical history; or combinations thereof. The profile defines personal input data, which is then used to further calibrate the computer-based model of drug response.

One embodiment of the present invention provides a sophisticated platform for the analysis of pharmacodynamic, pharmacokinetic outcomes, drug-drug interactions (so-called DDIs) and tissue-specific responses in a given individual, resulting in a comprehensive personalised model. Such models can comprise nested compartments that represent different tissue functionalities and cell types within an organ system. When assembled, the levels of hierarchical complexity allow for modelling of molecularly-driven events, such as specific metabolic pathways. The blood flows and partition coefficients that link the compartments—e.g. the organ systems—together mathematically are estimated from animal, in vitro data, and clinical data. The parameters and compartments are then optimized to fit the model to existing data.

Hence, the present invention provides a significant advantage over and enhancement of prior art modelling systems that are largely based upon pharmacokinetic focused population level data, derived from animal or entirely in vitro based responses. In contrast, according to specific embodiments the present invention provides a virtual mimic, also referred to as a “Virtual Twin”, for an individual. This Virtual Twin may represent an in silico model that is configured so as to represent an entirely personalised model for a given individual. The model may represent the consolidation of multiple data inputs from a variety of sources. This approach facilitates the growth of personalised medicine solutions, improved design of dosage regimens and the identification of potentially harmful side effects before a drug, xenobiotic, or combination of same is administered.

The virtual simulator may also incorporate an in vitro to in vivo extrapolation (IVIVE) approach to further inform the model. The IVIVE approach establishes virtual populations by building up mechanistic and physiologically based pharmacokinetic (PBPK) models. These models incorporate identified variabilities in demographic and biological (genetic and environmental) components linked to drug-specific physicochemical properties (for example, aqueous and lipid solubilities) and in vitro data on absorption, metabolism and transport. The covariate relationships embedded in such models can be complex and nonlinear and can be difficult to resolve by simple linear covariate analysis. The primary advantage of the IVIVE approach is that it maximizes the value of all in vitro information previously generated during drug discovery and preclinical development.

The algorithm of an embodiment of the invention may include consideration of data derived from mRNA analysis, such as gene expression data for pharmacodynamic markers, may be categorised further via one or more additional gene and non-gene expression parameters, which may be derived from analysis of biomarkers detected in one or more biological samples. Non-gene expression parameters may include physiological and epidemiological information. In some embodiments of any aspects of the invention, one or more non-gene expression parameters may be selected from the group consisting of: ethnicity; genotype; age; age group classification; gender; smoking status; presence of chronic disease, including renal impairment, diabetes or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; or combinations thereof.

In a specific embodiment of the invention, the described methods can be implemented via one or more computer systems. According to a further embodiment, an apparatus comprising one or more memories and one or more processors is provided, wherein the one or more memories and the one or more processors are in electronic communication with each other, the one or more memories tangibly encoding a set of instructions for implementing the described methods of the invention. In another embodiment the invention provides a computer readable medium containing program instructions for implementing the method of the invention, wherein execution of the program instructions by a controller comprising one or more processors of a computer system causes the one or more processors to carry out the steps as described herein. Suitably, the data may be stored in a database, and accessed via a server. Suitably, the server is provided with communication modules to receive and send information, and processing modules to carry out the steps described herein. In some embodiments, the data is provided through a cloud service. In particular embodiments, the method is accessible as a web service. In some embodiments, users may access the service for recordal or retrieval of scores via a website, in a browser. Networking of computers permits various aspects of the invention to be carried out, stored in, and shared amongst one or more computer systems locally and at remote sites. Hence, two or more computer systems may be linked using wired or wireless means and may communicate with one another or with other computer systems directly and/or using a publicly available networking system such as the Internet.

Suitably, the computer system includes at least: an input device, an output device, a storage medium, and a microprocessor). Possible input devices include a keyboard, a computer mouse, a touch screen, and the like. Output devices computer monitor, a liquid-crystal display (LCD), light emitting diode (LED or OLED) computer monitor, virtual reality (VR) headset and the like. In addition, information can be output to a user, a user interface device (e.g. tablet PC, mobile phone), a computer-readable storage medium, or another local or networked computer. Storage media include various types of memory such as a hard disk, RAM, flash memory, and other magnetic, optical, physical, or electronic memory devices. The microprocessor is a computer microprocessor (e.g. CPU) for performing calculations and directing other functions for performing input, output, calculation, and display of data. In one embodiment of the invention, the computer processor may comprise an artificial neural network (ANN). In a further embodiment of the invention the computer processor may comprise a machine learning algorithm, suitably a machine learning algorithm that has been trained against one or more appropriate data sets.

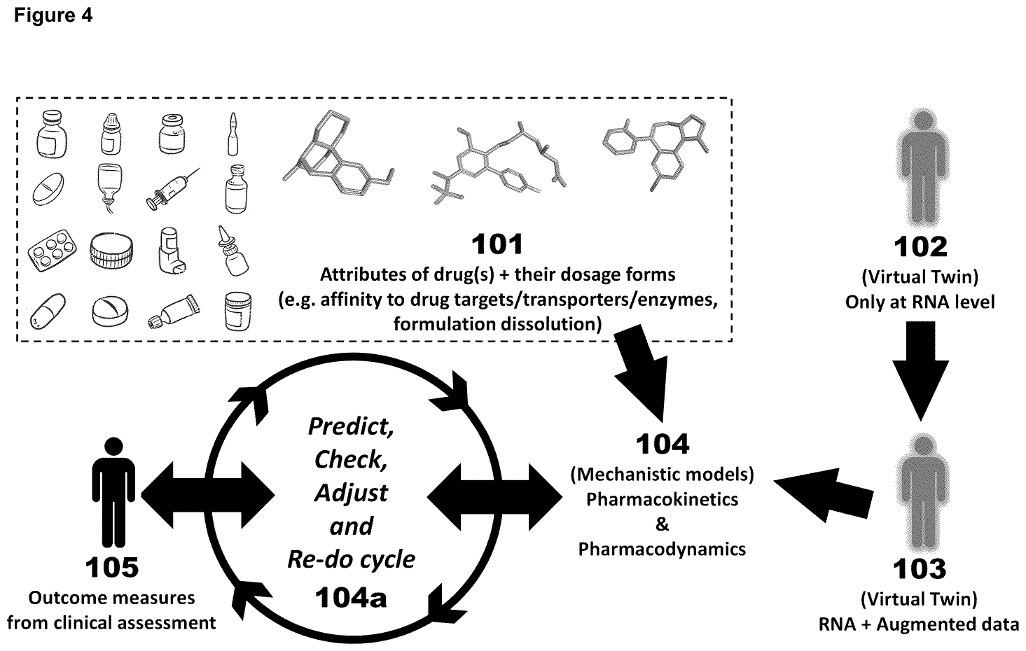

The modelling platform of the invention allows for accurate in silico simulation of pharmacodynamic and pharmacokinetic responses by combining two primary classes of data and is summarised in FIG. 4. The first class of data 102 is the first input data in the form of circulating mRNA expression for pharmacodynamic markers associated with a defined pathophysiology, and any augmented information 103 (as described above) related to the individual. The second class of data 101 is termed “second input data” and relates to the identity of the drug, compound or substance under test as well as attributes of these molecules and any impact of the choice of dosage as well as formulation (e.g., affinity to drug targets/transporters/enzymes, bioavailability and/or formulation dissolution kinetics). If drug-drug interactions (DDI) are under consideration then there may be a plurality of second input data. These two types of data may be conveniently stored within XML-based or JavaScript-based file format that can be viewed and accessed via the system graphical user interface (GUI) as well as other tools such as Microsoft Edge™ (Microsoft Corp., Redmond (WA), USA) or Google Chrome (Google LLC, Mountain View (CA), USA). The schema of these files is designed to allow forward compatibility of files over time such that future release versions and new parameters may be added without disrupting what already exists. This allows files created with a current version of the simulator and to be used with later versions when they are released where any possible missing values are automatically replaced with default values. Files may contain a degree of meta data showing varying information including the software version used to create the file.

The first input data 102 and second input data 101 provides the baseline information for initiating an output model simulation of drug response for a given individual 105 leading to the determination of appropriate clinical outcome decisions. However, it may also be necessary or desirable to create a workspace 104 that provides contextual information about conditions in which the trial is to be undertaken as well as including mechanistic models of pharmacokinetics (e.g. PBPK and/or Population PK modelling) and pharmacodynamics. The workspace file may also be XML or JavaScript-based; however, this time it acts as a container for first and second input data 102, 101 as well as any trial/simulation modelling information and applied user defined settings. The workspace 104 may also be used as a snapshot of the running condition of any simulation. In other words, to reproduce any simulation exactly, all that is needed is a copy of the workspace 104 taken at the time the simulation was run. The model simulations can be iterated 104a against a range of clinical assumptions and rechecked for accuracy several times if required. In this phase, the modelling platform may be adjusted and re-tested to accommodate for unexpected predicted DDI, or poor clinical outcomes based upon the input information (e.g., drugs or formulations) selected.

Hence, the output of the virtual in silico model 105 of an embodiment of the invention comprises algorithms that are able to incorporate in vitro and in vivo data on drug response with inter-individual variability that is relevant to the tissues/compartment of the individual concerned. This allows for liquid biopsy transcriptomic data for a given individual to contribute to a virtual prediction of response to a proposed drug regimen.

Accordingly, in a specific embodiment of the invention a dosage regimen is provided, in which parameters related to the administration of a drug comprising a pharmaceutical compound or a biological therapeutic agent to a subject are determined in conjunction with that individual's pharmacodynamic response profile for the compound or agent. More specifically, a liquid biopsy may be obtained from a subject and cfRNA_{TOTAL} analysis performed. From the cfRNA_{TOTAL} analysis the presence of one or more pharmacodynamic markers is determined. The one or more pharmacodynamic markers may include a drug target.

In an embodiment of the invention the pharmacodynamic markers of disease that are suitably identified from cfRNA_{TOTAL} analysis may be one or more of the markers listed in Table 1 below, or similar markers of disease, organ function or drug effect.

TABLE 1
Examples of pharmacodynamic and disease markers and
the type of liquid biopsy in which they are measured.

| | | Liquid biopsy |
|---|---|---|
| Marker | Disease/condition | sample |
| Alanine transaminase (GPT) | Liver disease | Blood |
| Aspartate aminotransferase | Liver damage | |
| (GOT1/2) | | |
| Alkaline phosphatase (ALPL) | Liver function | |
| Albumin (ALB) | | |
| C-reactive protein (CRP) | Inflammation | Blood |
| | Bacterial infection | |
| | Inflammatory bowel syndrome | |
| | Autoimmune disorders | |
| Albumin (ALB) | Kidney disease | Urine |
| Creatinine | Kidney function | |
| Hemoglobin | Anemia | Blood |
| Hematocrit | Iron deficiency | |
| High-density lipoprotein (HDL) | Heart disease | Blood |
| Low-density lipoprotein (LDL) | Obesity | |
| Creatine phosphokinase 1 (CPK-1) | Brain injury | Blood |
| Creatine phosphokinase 2 (CPK-2) | Myocardial infarction | Blood |
| Creatine phosphokinase 3 (CPK-3) | Heart injury | |
| Troponin | | |
| Prostate-specific antigen (PSA) | Prostate cancer | Blood |
| Cancer antigen 125 (CA125) | Ovarian cancer | Blood |
| Calcitonin | Medullary thyroid cancer | Blood |
| Alpha-fetoprotein (AFP) | Liver cancer | Blood |

In further embodiments of the invention the pharmacodynamic markers of disease are suitably identified from cfRNA_{TOTAL} that is obtained from the exosomal content from human plasma. Table 2 (below) provides pharmacodynamic markers that are associated with specific pathologies that may be involved in a range of diseases and disease states.

TABLE 2
List of disease and pharmacodynamic markers measured in plasma exosomes. The list includes tyrosine
protein kinases, other kinases (serine/threonine-protein kinases), growth factors, markers involved
in immunity regulation and immune response, markers involved in apoptosis, inflammation and angiogenesis
and markers related to organ/system health and homeostasis. Underlined markers have a confirmed
link to disease and those in bold font are FDA-approved drug targets.

Tyrosine kinases (cell cycle, proliferation, differentiation, migration and death)
AATK, ABL1/2, AXL, BLK, BMX, BTK, CSF1R, CSK, DDR1, DDR2, EGFR, EPHA1/2/3/4/5/6/7/8/10,
EPHB1/2/3/4/6, ERBB2/3/4, FER, FES, FGR, FLT1/3/4, FRK, FYN, HCK, IGF1R, INSR, INSRR, JAK1/2/3,
KDR, KIT, LCK, LMTK2/3, LTK, LYN, MATK, MERTK, MET, MUSK, NTRK1/2/3, PDGFRA/B, PKDCC,
PTK2/2B/6/7, RET, ROS1, RYK, SRC, SRMS, STYK1, SYK, TEC, TEK, TIE1, TNK1/2, TXK, TYK2,
TYRO3, YES1, ZAP70
Other protein kinases (cell cycle, proliferation, differentiation, migration and death)
ILK, MAP3K5, MAPK1, MAPK3, MAPK4, MAPK6, MAPK7, MAPK8, MAPK9, MAPK10, MAPK11,
MAPK12, MAPK13, MAPK14, MAPK15, PAK1/2/3/4/5/6/7, PLK1/2/3/4, PRKCA, PRKCB, PRKCD,
PRKCE, PRKCG, PRKCH, PRKCI, PRKCQ, PRKCZ, TGFBR1, TNNI3K, ZAK
Growth factors and receptors
ABL1/2, EGFR, ERBB2/3/4, FGF19/21, FGFR1-4, GNB2L1, RYK
Immunity regulation and immune response
CXCR1/2/4/5/6/7, DPP4/9, IFITM1/2/3/10, IL1B/F10, IL2, IL3, IL4, IL4I1, IL5, IL6, IL6ST, IL7, IL8, IL9, IL10,
IL11, IL12A/B, IL13, IL15, IL16, IL17A/B/C/F, IL18, IL19, IL21, IL22, IL23A, IL25, IL26, IL27, IL28A/B, IL31,
IL32, IL33, IL34, IL36A/B/G, IL37, IL1R1/2, IL1RAPL1/2, IL1RL1/2, IL1RN, IL2RA/B/G, IL3RA, IL4R,
IL5RA, IL6R, IL7R, IL9R, IL10RA/B, IL11RA, IL12RB1/2, IL13RA1/2, IL15RA, IL17RA/B/C/D/E/EL,
IL18R1, IL18RAP, IL20RA/B, IL21R, IL21R-AS1, IL22RA1/2, IL23R, IL27RA, IL28RA, IL31RA, ILF2, ILF3,
IRAK4, ITK, LGALS2/3/8/9, MST1R, NR1H4, PDCD1, TLR1/2/3/4/5/6/7/8/9/10
Apoptosis
CASP1-10/14, DPP8, GNB2L1, LGALS1/4/7B/9B/9C/12/14/17A, LGALSL, PDCD1
Organ health and homeostasis
ACACA/B, AGTR1, AGTRAP, ALPI, ALPL, ALPP, CASP14, CAV1/2, CAVIN1/2, CKB, CKM,
CKMT1A/1B/2, DPP3/4/6/8/9/10, ESR1, GLP1R, GOT1/2, GPBAR1, GPT, GPT2, GPX1/2/3/4/7/8, GSG2,
GSR, HMGCR, LOXL1/2/3/4, MC4R, MSH2, NPC1L1, NPR1/2/3, NR3C2, NR5A2, P4HB, PDIA3/4/5/6,
PGR, PNPLA3/6, PPARA, PPARD, PPARG, SCD, SERPINH1, STRAP,
TEX2/9/10/11/14/15/19/22/26/28/29/30/33/35/101/261/264, THRB, TMX3, TNC
Inflammation regulation and inflammatory response
CASP1/4/5/12, LOXL3, NR1H4, TLR1/2/3/4/5/6/7/8/9/10
Angiogenesis
ANGPTL1-7, AOC2/3, TNC, VEGFB, VEGFC

It will be appreciated that having information of this type for any given individual in relation to a proposed pharmaceutical compound or a biological therapeutic agent that is to be administered enables a precision dosing regimen to be formulated for that individual for the specific drug.

The systems of the present invention may perform some or all steps of the methods of the invention under the control of a processor. Hence, the system can comprise one or more processors and one or more computer-readable storage media. The computer readable storage media can have stored thereon computer-executable instructions that are executable by the one or more processors to cause the computer system to perform the methods and procedures described herein. Any of the steps, operations, methods or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, methods or processes described. By way of example, process steps may be carried out via use of laboratory automation protocols utilising one or more robotic systems or liquid handling devices. Such systems and devices may comprise analytic modules or functionality including plate readers for detection of reactions involving absorbance, fluorescence intensity, luminescence, time-resolved fluorescence, and/or fluorescence polarization. Examples of liquid handling systems suitable for the performance of automated laboratory protocols based upon the methodology described herein may include Freedom EVO (Tecan), Fluent (Tecan), JANUS® (PerkinElmer), Biomek® (Beckman Coulter), Microlab STAR® (Hamilton Robotics), Microlab VANTAGE® (Hamilton Robotics), EpMotion® (Eppendorf), Echo® (LabCyte), Mosquito® (TTP Labtech), OT-1 and OT-2 (Opentrons), LYNX® (Dynamic Devices), PIPETMAX® (Gilson), and Bravo (Agilent).

Reporting of the output data from a modelling system of the invention may be achieved via the GUI or via an output file that may comprise a .csv file or spreadsheet, such as Microsoft Excel™ (Microsoft Corp., Redmond (WA), USA) or Google Sheets (Google LLC., Mountain View (CA), USA). By way of non-limiting example, when the reporting process is implemented through the Excel Automation interface which is based on the Office Object Model. The simulation platform uses this technology to create or connect to an Excel application Component Object Model (COM) object, to manipulate and add worksheets as required. Each worksheet is a bespoke output based on the simulation input selections: each cell is effectively created individually with the selection of font (including size and weight), colour (both foreground and background), alignment of text within the cell, number format (based on the users' machine selection) as well as many other specifications.

After the output data has been rendered, graphical representations, such as dashboards, charts, pictograms or graphs may are added if applicable. These may include pharmacodynamic marker concentration-time profiles or, for example, pie charts which are created based on the output data comprised within the worksheet and formatted individually based on user selections such as number format, dashboard arrangement and also the colour ‘skin’ chosen before displaying the data.

In an alternative embodiment of the invention output data is comprised within a relational database. An advantage of this embodiment is that a simulator algorithm may be comprised as part of an organisational workflow as it can then write directly into a corporate database, for example. This enables formatting and visualisation and data analytics to be customised by the user.

Embodiments of the invention may also relate to an apparatus or device for performing a set of operations as defined herein, such as a set of operations that may suitably implement at least one embodiment of the present invention. The apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of medium suitable for storing electronic instructions, which may be coupled to a computer system bus. Furthermore, any computing systems referred to in the specification may include a single processor or may be architectures employing multiple processor designs for increased computing capability.

In a specific embodiment of the invention, a computer implemented process is provided for generating a pharmacodynamic profile or model for an individual subject who is suffering from a given disease or pathological condition. In this instance, the disease or pathology will be associated with one or more known pharmacodynamic marker genes whose expression may increase or decrease as a result of the disease or condition, either systemically or within a particular compartment of the body. Likewise, a treatment for the disease, such as a chemotherapy, is also likely to be associated with one or more known pharmacodynamic markers. Hence, there is an initial step of pharmacodynamic marker selection for a given disease. Suitably, this at least first pharmacodynamic marker is a gene that is expressed as a first mRNA and, it should be a gene that is likely to be detectable as cfRNA within a liquid biopsy. The process involves isolating the total cell free RNA (cfRNA_{TOTAL}) from a liquid biopsy obtained from the individual subject and then analysing the cfRNA_{TOTAL} to determine an amount of the first pharmacodynamic mRNA present in the liquid biopsy. This allows for determination of an amount (i.e. a level) of the first pharmacodynamic marker in the individual subject which may contribute to the generation of the profile or model. If two, three or more pharmacodynamic markers are detected and quantified the levels of these markers also may contribute to the generation of the pharmacodynamic profile or model. Where the markers are derived from a particular origin, such as a compartment or organ within the body of the subject, normalisation such as via shedding correction may be applied to enable accurate determination of the level of the given pharmacodynamic marker(s). The process may be carried out at a single time point or over a period of time, thereby enabling pharmacodynamic profiles to be built that track the course of a disease treatment, for example, for a specified individual. In addition, data from individuals may be combined to create population based models that can be used to inform clinical trials or dosage recommendations.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

The invention is illustrated by the following non-limiting examples.

### EXAMPLES

### Example 1

The following example provides a protocol for total RNA extraction from samples of blood that can be used to determine the levels of RNA for drug metabolizing enzymes, transporters and/or tissue-specific marker genes in the samples, and/or RNA for the determination of pharmacodynamic markers. Methods for the isolation of total protein and quantification of markers are described herein for the assessment of correlation between plasma RNA and tissue protein levels.

### RNA Analysis of Liquid Biopsy Comprising Blood

### Blood Samples

A liquid biopsy consisting of fresh peripheral venous blood may be collected from a subject and plasma isolated before further processing as described below. If required, peripheral blood mononuclear cells (PBMCs), including B and T lymphocytes, may be isolated using Ficoll-Paque PLUS (GE Healthcare Life Sciences).

Isolated plasma is stored frozen −80° C. until used for cell free RNA (cfRNA) isolation and measurement. Isolation of circulating or exosomal RNA can be done using a suitable RNA extraction kit such as the Qiagen QIAamp Circulating Nucleic Acid Kit as per the manufacturer's instructions (Qiagen, Hilden, Germany). Total nucleic acid is collected by such kits, and DNA is removed using a suitable kit such as the Qiagen RNase-free DNase Set or Ambion Turbo DNA-free Kit (Life Technologies, Carlsbad, California, USA). Eluted RNA, after DNA removal, is then detected as a quality control using a suitable total nucleic acid assessment technique such as the Agilent RNA pico Kit on Bioanalyzer equipment (Agilent Technologies, Eugene, Oregon, USA). RNA of sufficient quality is then stored for subsequent quantification.

### Reverse Transcription-PCR and RNA Sequencing

RNA (5-10 ng) may be reverse-transcribed using M-MLV Reverse Transcriptase (Invitrogen, Life Technologies, Inc.). Samples are amplified with PCR in a final reaction volume of 25 μl containing 2.5 μl of 10 times buffer, 0.1 μl of 10 mM dNTPs, 10 pmoles of each primer and 0.5 units of Taq DNA Polymerase. To confirm the presence and integrity of the cDNA template, the housekeeping gene, GAPDH, is amplified for each sample using primers GAPDH-5 (5′-ACCACAGTCCATGCCATCAC-3′; SEQ ID NO: 1) and GAPDH-3 (5′-TCCACCACCCTGTTGCTGTA-3′; SEQ ID NO: 2). Conditions may be as follows: an initial denaturation step for 5 minutes at 94° C., then 50 seconds at 94° C., 45 seconds at 55° C., and 1 min at 72° C. for 30 cycles, followed by an elongation step for 10 minutes at 72° C.

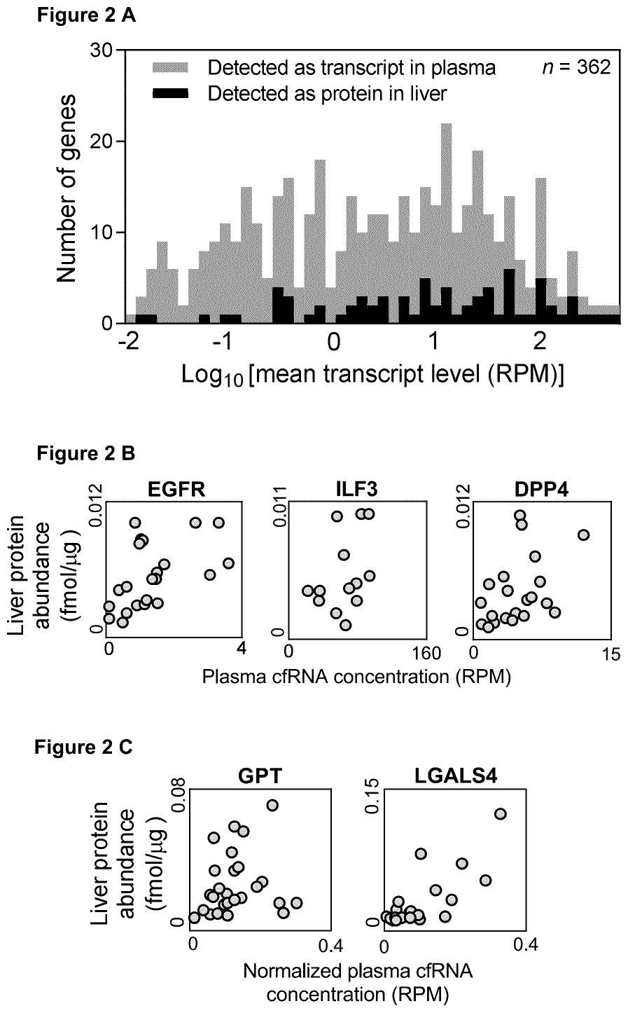

The cDNA obtained from the extracted total RNA may be analysed further, such as via a DNA microarray, in order to determine the identities and expression levels of genes expressed within the PBMCs or plasma samples. Alternatively, reverse transcription and amplification can be performed using a suitable genome sequencing method, such as Ampliseq (Life Technologies, ThermoFisher, Austin, TX). Up to 20,000 genes can be sequenced simultaneously and several libraries (one library per sample) can be analysed in one experiment. As an example, determination of the expression of more than 360 pharmacodynamic markers related to cancer biology, immune response and liver health was made in a liquid biopsy (FIG. 2A). Other genes which may be determined include marker genes specific for hepatic tissue, used to determine liver-specific shedding for the particular individual (see Example 2 below).

The above protocol may be repeated as necessary for multiple individuals in order to generate data on the expression of pharmacodynamic markers and/or the expression of organ-specific marker genes. The data are suitable for interrogation via bioinformatics techniques to determine correlations between marker expression as circulating RNA and expression as protein in tissue. The correlations are used to develop a virtual model of xenobiotic pharmacology that can be configured on a person by person basis in order to provide a virtual twin model of compound clearance and effect within a given individual with a particular disease.

### Example 2

### Accounting for Organ Specific Shedding

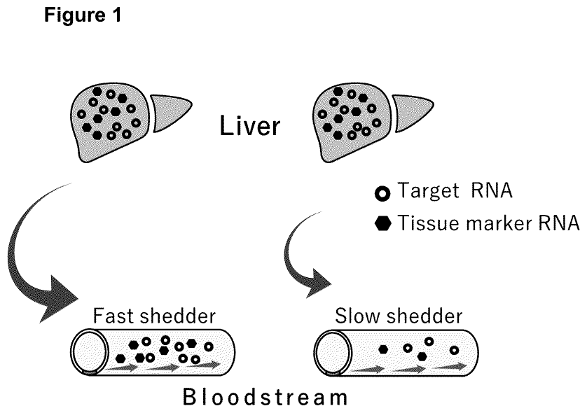

The following example provides a protocol for determining the degree of RNA shedding from liver tissue of a particular individual subject to the bloodstream, so establishing a robust and significant correlation function between hepatic protein levels and the corresponding plasma RNA concentrations of liver-specific pharmacodynamic markers. FIG. 1 shows a graphical representation of RNA shedding from liver into the blood. The targets are the RNA species of interest, including enzymes, transporters and pharmacodynamic markers. Liver-specific markers are used to monitor shedding in each individual specific to the liver. This shedding characterization can also be applied to other organs, including, but not limited to, the intestine, kidneys, heart, brain and lungs; as well as other bodily fluids.

A set of genes expressed principally in the liver were selected and a panel of nucleic acid primers specific to their sequences were used to assess their expression levels in 29 plasma samples from liver cancer patients (13 female, age range 44-85 years). Five Samples from healthy individuals (2 female, age range 23-57 years) were also processed in three technical replicates (n=5×3). These genes were selected for being specifically expressed in the liver, at significantly high levels to be considered representative of liver shedding. Among the list of these genes, 13 that were consistently detected in plasma samples were used as liver-specific plasma markers (Table 3), which together are proposed to make up a liver shedding factor (SF). This correction factor is calculated as the mean expression level of one or more of these genes in plasma, assessed using the same quantitative transcriptomic methodology (e.g. a gene sequencing technique or quantitative RT-PCR). This rate of RNA shedding and associated inter-individual variability used for the normalization of expression in plasma are proposed to be a part of the correlation function with protein expression.

Table 3 shows the panel of marker genes, and the detection reproducibility of sequencing data of the markers RNA in plasma samples. Data are expressed as percentage of replicates where the marker sequences were detected. Genes: A1BG (Alpha-1-B glycoprotein), FGB (Fibrinogen beta chain), AHSG (alpha-2-HS-glycoprotein), APOA2 (Apolipoprotein A-II), CFHR2 (Complement factor H-related 5), F2 (Coagulation factor II (thrombin)), F9 (Coagulation factor IX), HPX (Hemopexin), SPP2 (Secreted phosphoprotein 2), TF (Transferrin), C9 (Complement component C9), MBL2 (Mannose-binding protein C) and SERPINC1 (Antithrombin-III).

TABLE 3
Detection reproducibility of circulating marker RNA in human plasma. Data are expressed as a percentage.

| Genes | APOA2 | FGB | AHSG | HPX | SERPINC1 | F2 | CFHR2 | F9 | SPP2 | MBL2 | A1BG | TF | C9 |
|---|---|---|---|---|---|---|---|---|---|---|---|---|---|
| Cancer | 100 | 100 | 100 | 90 | 93 | 93 | 100 | 93 | 79 | 76 | 90 | 97 | 83 |
| plasma | | | | | | | | | | | | | |
| Healthy | 100 | 100 | 100 | 100 | 100 | 100 | 100 | 60 | 80 | 60 | 40 | 80 | 60 |
| plasma | | | | | | | | | | | | | |

Inter-individual variability in the expression of these markers highlights the presence of different degrees of shedding between individuals. Calculating this factor based on expression of 13 genes should assist in offsetting technical variability inherent to quantifying each of the genes individually, however it is contemplated that use of one or more of these genes or others would also be of use in carrying out the invention. Using preliminary data, inter-individual variability in liver SF was estimated at 32% in healthy plasma (n=5), calculated as percent coefficient of variation (% CV=100×SD/X̅), representing up to 2-fold variability between individuals. This illustrates by how much quantification of mRNA coding for drug clearance proteins or for pharmacodynamic markers may need to be scaled in order to correct for individual variability in RNA shedding rates. In the cancer cohort, however, this level of variability increased up to 84% (n=29), representing 77-fold variability between cancer patients. Table 4 gives details of the cancer patients from whom samples were collected.

TABLE 4
Demographics of cancer patients

| | | | Body | |
|---|---|---|---|---|
| Donor | | | mass | |
| code | Sex | Age | index | Diagnosis |
| 662 | F | 76 | — | Gall bladder cancer |
| 697 | M | 66 | 34 | Colorectal liver metastasis |
| 728 | M | 69 | 26 | Colorectal liver metastasis |
| 746 | M | 85 | 24 | Colorectal liver metastasis |
| 766 | F | 72 | — | Colorectal liver metastasis |
| 794 | F | 71 | 22 | Colorectal liver metastasis |
| 806 | M | 56 | 20 | Colorectal liver metastasis |
| 813 | F | 60 | 42 | Colorectal liver metastasis |
| 818 | M | 58 | 22 | Colorectal liver metastasis |
| 829 | M | 78 | 24 | Colorectal liver metastasis |
| 855 | F | 79 | 19 | Hepatocellular carcinoma |
| 1071 | M | 71 | — | Colorectal liver metastasis |
| 1304 | M | 57 | 23 | Colorectal liver metastasis |
| 1372 | F | 65 | 21 | Colorectal liver metastasis |
| 493 | F | 50 | — | Colorectal liver metastasis |
| 590 | M | 72 | 32 | Colorectal liver metastasis |
| 645 | F | 61 | 36 | Colorectal liver metastasis |
| 646 | M | 44 | 30 | Colorectal liver metastasis |
| 674 | F | 68 | 27 | Colorectal liver metastasis |
| 682 | M | 60 | 33 | Colorectal liver metastasis |
| 756 | M | 57 | 27 | Colorectal liver metastasis |
| 78 | M | 57 | 31 | Colorectal liver metastasis |
| 734 | F | 64 | 24 | Colorectal liver metastasis |
| 755 | M | 63 | 28 | Colorectal liver metastasis |
| 770 | F | 61 | 21 | Colorectal liver metastasis |
| 389 | F | 52 | 31 | Colorectal liver metastasis |
| 589 | F | 69 | 21 | Colorectal liver metastasis |
| 1063 | M | 77 | 27 | Hepatocellular carcinoma |
| 1359 | M | 68 | 33 | Hepatocellular carcinoma |

The proposed assessment should take into account the variability of shedding, with the proposed correction being applied to expression levels in plasma as follows (e.g. using 13 markers).

$$SF = {10}^{6} \cdot {\sum }_{i = 1}^{13} {[ cf RNA ]}_{{Marker}_{i}} / 13$$

$${( {[ cf RNA ]}_{Target} )}_{Normalized} = {[ cf RNA ]}_{Target} / SF$$

The outcome should be a normalized reading for each enzyme expressed per million reads (RPM) in a plasma sample of specified volume (1-5 ml).

### Example 3

### Quantification of Pharmacodynamic Markers in Tissue Samples

For the quantification of a large number of proteins in tissue samples, a global proteomic approach is adopted which allows simultaneous detection and measurement of several disease and drug effect related markers. This approach also enables quantification of drug metabolizing enzymes and transporters, which facilitates creation of realistic virtual simulations as part of IVIVE-PBPK models. In particular, the expression and activity of pharmacokinetic and pharmacodynamic markers are correlated and correlations affecting drug absorption, distribution, metabolism, excretion and drug effect are used to inform the creation of in silico models.

Human liver samples obtained from individual patients may be used to determine the levels of enzymes, transporters and pharmacodynamic markers by standard quantitative techniques such as Western blot or ELISA. A biopsy of liver tissue is taken from the same subject who supplied the blood sample, to provide a matched set. The tissue is physically homogenized using either a manual device (such as a ground glass Ten Broeck tissue grinder) or a mechanical/powered tissue homogenizer (e.g. a Tekmar Tissuemizer) in the presence of an appropriate extraction buffer in order to obtain a homogeneous suspension. Differential centrifugation may be used to extract relevant fractions that can be analysed to measure protein or RNA, including homogenates, S9 fractions, cytosols and crude/microsomal membrane fractions.

### Sample Preparation

Total membrane or microsomal protein concentration may be determined in triplicate using a colorimetric assay, such as the Lowry, Bradford or BCA assay. Sample preparation may follow a gel-based (Achour et al. 2014, Drug Metab. Dispos. 42, 500-510), solution-based (Harwood et al., 2015, Pharm. Biomed. Anal. 110, 27-33) or filter-aided method (Wisniewski et al., 2009, Nat. Methods 6, 359-362). Samples are prepared in a suitable buffer, such as 50 mM ammonium bicarbonate (˜pH 8.0), normally with reduction (for example, with dithiothreitol) and alkylation (for example, with iodoacetamide) of protein disulfide bridges. Protein digestion strategies use different enzymes, mainly mammalian or recombinant trypsin. Sequential digestion strategies should afford better digestion efficiency. Proteins are digested to peptides in a suitable buffer (e.g. 50 mM ammonium bicarbonate), firstly with lysyl endopeptidase (1-2% w/w) at 30° C. for 3-4 hours, followed by trypsin (1-5% w/w) at 37° C. for 10-18 hours. Standard methods for peptide desalting, cleaning and collection can be applied. Sample volume can be reduced down to 50 μl, or lower volume, using a vacuum concentrator and volume can be adjusted with buffer, and stored at −20° C.

### Mass Spectrometry Analysis of Sample Peptides

Sample peptides are analysed by LC-MS/MS using suitable LC-MS/MS equipment [nano-HPLC system (e.g. nano-Acquity nanoUPLC system, Waters, UK) coupled to an Orbitrap mass spectrometer (e.g. Orbitrap Elite mass spectrometer, ThermoScientific, Pittsburgh, PA)] in data-dependent mode. Data are acquired by software used for operating the mass spectrometer (e.g. Xcalibur, Thermo Fisher). Samples (1 μl) are injected either directly onto an analytical column or onto a trapping column connected to an analytical column at a nanoflow rate using a suitable low-to-high acetonitrile gradient.

### Data Analysis of the Measured Proteins

The abundance of each enzyme in the sample may be calculated using the total protein approach (Wisniewski 2017, Methods Enzymol., 585: 49-60). The abundance data are measured in units of μmol/mg microsomal protein and thereafter converted to fmol/μg liver tissue using the microsomal protein per gram liver (MPPGL) scalar (mass of total membrane protein per unit tissue measured using the Lowry, Bradford or bicinchoninic acid assay).

### Example 4

### Correlation Between Plasma RNA and Tissue Protein Levels of Pharmacodynamic Markers

Pharmacodynamic and disease markers can be either ubiquitously expressed (present in multiple organs or compartments) or specific to a small number of tissues or a single tissue only, for example the liver. The correlation can be applied with or without correcting the cfRNA data with an organ-specific SF, as shown in Example 2. Correction for shedding in a specific organ is applicable with tissue or group (of tissues) enriched targets. In this present example, correlation analysis is performed for hepatic protein levels and plasma mRNA levels reported above the limit of quantification.

FIG. 2B shows the measured plasma levels of three ubiquitously expressed pharmacodynamic markers in liver cancer patients correlated to the quantified protein abundance levels of these markers in matched organ tissue (liver). The RNA levels in plasma were not normalized to shedding because these markers originate from multiple organs (compartments) including the liver. Instead, normalization was carried out to total cfRNA detected in plasma. The three pharmacodynamic targets are: epidermal growth factor receptor (EGFR), interleukin enhancer-binding factor 3-A (ILF3), and dipeptide peptidase 4 (DPP4). EGFR is a prognosis factor for several cancer types (e.g. colorectal, liver and non-small cell lung carcinoma) and a therapeutic target with two existing drug classes: tyrosine kinase inhibitors (e.g. erlotinib and lapatinib) and monoclonal antibodies (e.g. cetuximab and panitumumab). ILF3 is involved in cancer development and immune response modulation and is useful as a cancer prognosis factor. DPP4 is implicated in development of cancer and diabetes mellitus and is used as a cancer prognosis factor and targeted by the anti-diabetic therapeutic class, DPP4 inhibitors (e.g. sitagliptin and linagliptin).

FIG. 2C shows the measured plasma expression levels of two tissue-specific pharmacodynamic markers in cancer patients correlated to the quantified protein abundance levels of these markers in matched organ tissue (liver). Plasma RNA levels have been adjusted to account for liver-specific shedding. The two pharmacodynamic markers are alanine aminotransferase 1 (GPT) and galectin 4 (LGALS4). GPT is used as a marker of liver function or liver disease. LGALS4 plays a role in apoptosis and immune response and is a prognosis marker of renal, bladder and hepatic cancers.

The experiments detailed herein confirm that determination of the amounts of circulating plasma mRNA can be used to assign the relative abundance of a plurality of proteins that determine the pharmacological effect, and that such quantification can be improved further when levels of organ-specific mRNA are adjusted using a SF, indicating a baseline level of cell free mRNA shedding. The approach taken in the present invention allows for the creation of in silico models that will permit the prediction of the effect of particular compounds in an individual subject.

### Example 5

Changes in Liquid Biopsy cfRNA Identify Potential Pharmacodynamic Markers of Disease

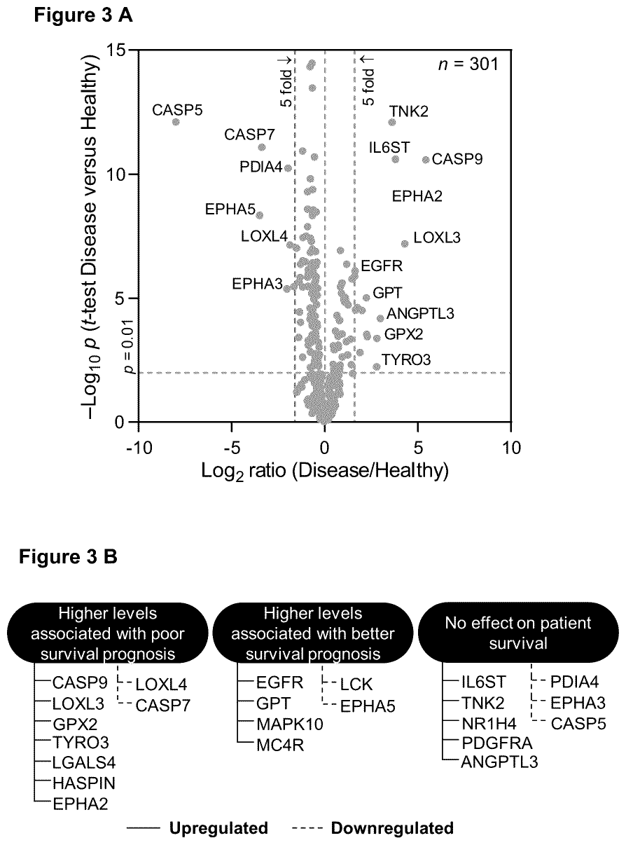

Differential expression of disease and pharmacodynamic markers is assessed for a disease state, including but not limited to cancer (e.g. liver cancer) relative to healthy plasma. Significantly upregulated and downregulated genes are identified. FIG. 3A shows differentially expressed pharmacodynamic and disease markers in plasma from liver cancer patients. In this instance sixteen pharmacodynamic markers were upregulated and seven were downregulated. Details of these markers are shown in Table 5, below. These markers are useful prognostic markers of patient survival as illustrated in FIG. 3B and several of these are drug targets, including but not limited to receptor tyrosine kinases.

TABLE 5
Changes in gene expression in cancer from
healthy baseline measured in liquid biopsy.
Upregulated in cancer cohort

| | Fold difference | p-value | |
|---|---|---|---|
| Gene | from healthy | (<0.01) | Function |
| CASP9 | 227.4 | 2.7 × 10⁻¹¹ | Pro-apoptotic |
| LOXL3 | 72.9 | 6.4 × 10⁻⁸ | Pro-metastasis |
| IL6ST | 44.8 | 2.5 × 10⁻¹¹ | Oncogene involved in immune response |
| TNK2 | 36.9 | 8.2 × 10⁻¹³ | Cell migration |
| EPHA2 | 25.0 | 3.1 × 10⁻¹⁰ | Receptor tyrosine kinase involved in |
| | | | proliferation and migration |
| ANGPTL3 | 19.6 | 6.6 × 10⁻⁵ | Homeostasis (lipid, cholesterol and ions) |
| GPX2 | 16.5 | 4.1 × 10⁻⁴ | Redox protection |
| TYRO3 | 16.0 | 5.8 × 10⁻³ | Receptor tyrosine kinase involved in |
| | | | survival, proliferation and migration |
| NR1H4 | 9.9 | 3.6 × 10⁻⁴ | Immune response |
| PDGFRA | 9.5 | 2.8 × 10⁻⁴ | Cell proliferation |
| GPT | 9.3 | 9.6 × 10⁻⁶ | Liver function/damage marker |
| MAPK10 | 7.4 | 3.2 × 10⁻⁵ | Neuronal proliferation/differentiation/ |
| | | | migration |
| LGALS4 | 6.5 | 1.6 × 10⁻³ | Tumor growth/progression |
| MC4R | 5.8 | 2.4 × 10⁻⁵ | Body-weight regulation/wasting |
| HASPIN | 5.2 | 3.0 × 10⁻⁵ | Cancer development and progression |
| EGFR | 5.0 | 8.0 × 10⁻⁷ | Growth factor, receptor tyrosine kinase |

Downregulated in cancer cohort

| | Fold difference | p-value | |
|---|---|---|---|
| Gene | from healthy | (<0.01) | Function |
| LCK | 0.19 | 3.5 × 10⁻⁶ | Pro-T-Cell immune response |
| LOXL4 | 0.15 | 7.2 × 10⁻⁸ | Tumor suppressor |
| PDIA4 | 0.14 | 5.7 × 10⁻¹¹ | Correct folding of proteins |
| EPHA3 | 0.13 | 4.2 × 10⁻⁶ | Inhibits cancer growth |
| CASP7 | 0.03 | 8.4 × 10⁻¹² | Pro-apoptosis |
| EPHA5 | 0.03 | 4.6 × 10⁻⁹ | Inhibits invasion and migration |
| CASP5 | 0.0003 | 7.9 × 10⁻¹³ | Inflammation regulation |

In conclusion, these Examples demonstrate that a liquid biopsy analysis can inform pharmacodynamic decisions based on plasma measurements of cell free RNA. The use of this approach facilitates the deployment of precise and effective dosage regimens (precision dosing), as an essential element of precision medicine.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

What is claimed is:

1. A method of treating an individual subject, wherein the individual is the intended recipient of a pharmaceutical treatment, the method comprising establishing a personalised pharmacodynamic profile for the individual subject prior to or during the treatment, the method comprising the steps of:
isolating total cell free RNA (cfRNATOTAL) from a liquid biopsy obtained from the individual subject;
identifying an amount of a first cell free RNA (cfRNA) present in the liquid biopsy, wherein the first cfRNA originates from a specified compartment within the body of the subject, and wherein the first cfRNA encodes a first protein from the compartment that has a pharmacodynamic activity relevant to the pharmaceutical treatment;
determining a pharmacodynamic activity relevant to the pharmaceutical treatment for the individual subject based upon the presence or absence, or a level of the protein within the specified compartment of the subject;
generating the personalised pharmacodynamic profile for the individual subject; and
treating the individual according to a dosage regimen for the pharmaceutical treatment that is optimized to the individual based upon their personalised pharmacodynamic profile.

2. The method of claim 1, wherein the pharmaceutical treatment comprises administration of a xenobiotic.

3. The method of claim 2, wherein the xenobiotic comprises a pharmaceutical agent.

4. The method of claim 1, wherein the liquid biopsy comprises a sample of a bodily fluid selected from one of the group consisting of: blood; serum; plasma; urine; saliva; semen; tears; sweat; lymphatic fluid; bile; cerebrospinal fluid; ascites; pleural effusion; stool; and a mucus secretion.

5. The method of claim 1, wherein the method comprises identifying a first and a second cell free RNAs (cfRNAs).

6. The method of claim 1, wherein the method comprises identifying a plurality of cfRNAs each corresponding to a different compartment protein.

7. The method of claim 1, wherein the individual subject has a disease selected from one or more of the group consisting of: cancer; liver disease; inflammatory disease; allergy; metabolic diseases, including metabolic deficiency; degenerative diseases, including neurodegenerative diseases; psychiatric disorders; infection, including chronic or acute infection from bacterial, viral, fungal or parasitic pathogens; autoimmune disease; kidney disease; anemia; heart disease; myocardial infarction; obesity; fibrosis; and traumatic brain or CNS injury.

8. The method of claim 1, wherein the first protein that has a pharmacodynamic activity is a marker of cancer.

9. The method of claim 8, wherein the first protein is a marker of tumour growth.

10. The method of claim 8, wherein the first protein is a marker of metastases.

11. The method of claim 8, wherein the cancer is selected from one or more of the group consisting of: carcinomas; leukemias; adenocarcinomas; gliomas; glioblastoma; brain metastases; multiple myelomas; renal clear cell carcinoma; prostate cancer; pancreatic adenocarcinoma; melanoma; metastatic melanoma; rhabdomyosarcoma; hepatocellular carcinoma; metastatic liver cancer; colon tumours; breast cancer; non-small cell lung cancer; oral tumours; colorectal cancer; gallbladder cancer; brain tumours; Ewing's sarcoma; bladder cancer; meningioma's; lymphoma; viral-induced tumours; Burkitt's lymphoma; Hodgkin's lymphoma; adult T-cell leukemia; lymphoproliferative disease; Kaposi's sarcoma; as well as MALT lymphoma; papillary thyroid carcinoma; cervical cancer; osteosarcoma; primary intra-ocular B-cell lymphoma; mesotheliomas; ovarian cancer; cervical cancer; head and neck cancer; small cell lung cancer; cancer of the oesophagus; stomach cancer; hepatobiliary cancer; cancer of the small intestine; rectal cancer; kidney cancer; bladder cancer; penile cancer; urethral cancer; testicular cancer; cervical cancer; vaginal cancer; uterine cancer; thyroid cancer; parathyroid cancer; adrenal cancer; pancreatic endocrine cancer; carcinoid cancer; bone cancer; skin cancer; retinoblastomas; non-Hodgkin's lymphoma; multicentric Castleman's disease or AIDS-associated cancer; primary effusion lymphoma; and neuroectodermal tumours.

12. The method of claim 1, wherein the first protein that has a pharmacodynamic activity is a marker of inflammatory disease.

13. The method of claim 12, wherein the inflammatory disease is selected from one or more of the group consisting of: asthma; keratitis; rhinitis; stomatitis; mumps; pharyngitis; tonsillitis; tracheitis; bronchitis; pneumonia; myocarditis; gastritis; gastroenteritis; cholecystitis; and appendicitis.

14. The method of claim 1, wherein the first protein that has a pharmacodynamic activity is a marker of an autoimmune disorder.

15. The method of claim 14, wherein the autoimmune disorder is selected from one or more of the group consisting of: chronic lymphocytic thyroiditis; hyperthyroidism; insulin-dependent diabetes mellitus; myasthenia gravis; chronic ulcerative colitis; pernicious anemia associated with chronic atrophic gastritis; Goodpasture's syndrome; pemphigus vulgaris; pemphigoid; primary biliary cirrhosis; multiple cerebrospinal sclerosis; acute idiopathic neuritis; systemic lupus erythematosus; rheumatoid arthritis; psoriasis; systemic vasculitis; scleroderma; pemphigus; mixed connective tissue disease; autoimmune hemolytic anemia; autoimmune thyroid disease; Crohn's disease; transplant rejection and ulcerative colitis.

16. The method of claim 1, wherein the first protein that has a pharmacodynamic activity is a marker of a psychiatric disorder.

17. The method of claim 16, wherein the psychiatric disorder is selected from one or more of the group consisting of: dementia and Mild Cognitive Impairment (MCI); addiction; reduced adherence; or non-compliance; with a medication regime; eye gaze-associated disorders; dysthymia; psychotic disorders such as schizophrenia; eating disorders such as Anorexia Nervosa and Bulimia Nervosa; sleep disorders; developmental dyspraxia; attention deficit hyperactivity disorder; Tourette's syndrome; and personality disorders.

18. The method of claim 1, wherein the first protein that has a pharmacodynamic activity is a marker of a neurodegenerative disease.

19. The method of claim 18, wherein the neurodegenerative disease is selected from one or more of the group consisting of: Alzheimer (or Alzheimer's) disease; Parkinson's disease (including Parkinson's disease dementia); multiple sclerosis; adrenoleukodystrophy; AIDS dementia complex; Alexander disease; Alper's disease; amyotrophic lateral sclerosis (ALS); ataxia telangiectasia; Batten disease; bovine spongiform encephalopathy (BSE); Canavan disease; cerebral amyloid angiopathy; cerebellar ataxia; Cockayne syndrome; corticobasal degeneration; Creutzfeldt-Jakob disease (CJD); diffuse myelinoclastic sclerosis; fatal familial insomnia; Fazio-Londe disease; Friedreich's ataxia; frontotemporal dementia or lobar degeneration; hereditary spastic paraplegia; Huntington disease; Kennedy's disease; Krabbe disease; Lewy body dementia; Lyme disease; Machado-Joseph disease; motor neuron disease; Multiple systems atrophy; neuroacanthocytosis; Niemann-Pick disease; Pelizaeus-Merzbacher Disease; Pick's disease; primary lateral sclerosis including its juvenile form; progressive bulbar palsy; progressive supranuclear palsy; Refsum's disease including its infantile form; Sandhoff disease; Schilder's disease; spinal muscular atrophy; spinocerebellar ataxia; Steele-Richardson-Olszewski disease; subacute combined degeneration of the spinal cord; survival motor neuron spinal muscular atrophy; Tabes dorsalis; Tay-Sachs disease; toxic encephalopathy; transmissible spongiform encephalopathy; Vascular dementia; X-linked spinal muscular atrophy; synucleinopathy; progranulinopathy; tauopathy; amyloid disease; prion disease; protein aggregation disease; and neurodegenerative movement disorders.

20. The method of claim 1, wherein the first protein that has a pharmacodynamic activity is a marker of fibrosis.

21. The method of claim 20, wherein the fibrosis is selected from one or more of the group consisting of: liver cirrhosis, as well as idiopathic pulmonary fibrosis; renal fibrosis; endomyocardial fibrosis; and arthrofibrosis.

22. The method of claim 1, wherein the first protein that has a pharmacodynamic activity is epidermal growth factor receptor (EGFR).

23. The method of claim 1, wherein the first protein that has a pharmacodynamic activity is interleukin enhancer-binding factor 3-A (ILF3).

24. The method of claim 1, wherein the first protein that has a pharmacodynamic activity is dipeptide peptidase 4 (DPP4).

25. The method of claim 1, wherein the first protein that has a pharmacodynamic activity is alanine aminotransferase 1 (GPT).

26. The method of claim 1, wherein the first protein that has a pharmacodynamic activity is galectin 4 (LGALS4).

27. The method of claim 1, wherein the first protein that has a pharmacodynamic activity is caspase 9 (CASP9).

28. The method of claim 1, wherein the first protein that has a pharmacodynamic activity is Lysyl oxidase homolog 3 (LOXL3).

29. The method of claim 1, wherein the pharmaceutical treatment comprises administration of a pharmaceutical agent and wherein the method further comprises establishing a pharmacokinetic profile for the individual subject.

30. The method of claim 29, wherein the pharmacokinetic profile is established by identifying an amount of a further cell free RNA (cfRNA) present in the liquid biopsy, wherein the further cfRNA originates from a specified compartment within the body of the subject, and wherein the further cfRNA encodes a protein from the compartment that has a pharmacokinetic activity relevant to the pharmaceutical treatment.

31. The method of claim 30, wherein the pharmacokinetic activity is selected from one or more of the group consisting of: cytochrome P450 monooxygenase enzymes (CYPs); membrane transport proteins; and transferases.
